(19) United States
(12) Patent Application Publication
(10) Pub. No.: US 2016/0045645 A1
(43) Pub. Date: Feb. 18, 2016

(54) BIODEGRADABLE POLYMER WITH ENHANCED PHYSICAL PROPERTIES INCLUDING STEREOCOMPLEX ORGANIC FILLER AND METHOD FOR PRODUCING THE SAME

(71) Applicant: KOREA INSTITUTE OF SCIENCE AND TECHNOLOGY, Seoul (KR)

(72) Inventors: Soo Hyun KIM, Seoul (KR); Youngmee JUNG, Seoul (KR); Jin Ik LIM, Seoul (KR)

(21) Appl. No.: 14/514,786

(22) Filed: Oct. 15, 2014

(30) Foreign Application Priority Data
Aug. 12, 2014 (KR) 10-2014-0104432

(51) Int. Cl. A61L 31/12; A61L 31/06; A61L 31/04; A61L 31/14

(52) U.S. Cl. CPC A61L 31/129; A61L 31/148; A61L 31/06; A61L 31/041; A61L 2300/604

(57) ABSTRACT

Disclosed is a biodegradable polymer whose physical properties are improved by the presence of a stereocomplex organic filler. According to exemplary embodiments, the biodegradable polymer including a stereocomplex organic filler can find application in various fields, including biodegradable materials and medical materials, where conventional biodegradable polymers are difficult to use due to their inherent problems such as poor heat resistance and low strength. Also disclosed is a method for producing the biodegradable polymer.

### CROSS-REFERENCE TO RELATED APPLICATIONS

This application claims priority under 35 U.S.C. §119 to Korean Patent Application No. 10-2014-0104432 filed on Aug. 12, 2014 in the Korean Intellectual Property Office, the disclosure of which is incorporated herein by reference in its entirety.

### BACKGROUND OF THE INVENTION

1. Field of the Invention

The present invention relates to a biodegradable polymer whose physical properties are improved by the presence of a stereocomplex organic filler, and a method for producing the biodegradable polymer.

2. Description of the Related Art

Polymeric materials are used as biomaterials. Such polymeric materials include natural polymers, synthetic polymers, non-degradable polymers, and degradable polymers. For synthetic polymers, the inherent chemical and physical properties of constituent monomers can be controlled in the course of synthesis. Due to this advantage, various compositions of synthetic polymers have been developed to impart biomaterials with desired characteristics according to their purpose of use. Some synthetic polymers are used mainly for medical applications. For example, polymethyl methacrylate (PMMA) is used in eye lenses, polyethylene (PE) having a molecular weight of 500,000 or more is used in catheters, polyethylene (PE) having a molecular weight of 2,000,000 or more is used in artificial hip joints, and polytetrafluoroethylene (PTFE) is used as a material for artificial vessels.

On the other hand, biodegradable polyesters have received attention as synthetic biodegradable polymeric materials for medical applications, including bioabsorbable surgical sutures, materials for drug delivery systems, tools for the treatment of bone fracture, and artificial skins. Such biodegradable polyesters include polyglycolic acid (PGA) as a polymer of an α-hydroxy acid, poly-ε-caprolactone (PCL) polymerized from polylactic acid (PLA) and a lactone, and poly(lactic acid-co-glycolic acid) (PLGA). Under these circumstances, there is a need to develop inexpensive biodegradable polymers with excellent physical properties that can replace disposable plastics to solve environmental problems. However, conventional biodegradable polymeric materials with low glass transition temperature should be imparted with heat resistance, which limits their application in various materials. Another problem of conventional biodegradable polymeric materials is their low mechanical strength.

The use of inexpensive inorganic additives is considered a typical approach to improve the physical properties of conventional biodegradable polymers, for example, in terms of heat resistance and mechanical strength. The inorganic additives are advantageous for enhancing the physical properties of conventional biodegradable polymers. However, the inorganic additives are left in the form of solid during processing and are very hard, causing wear of processing equipment. Another disadvantage of the inorganic additives is poor interfacial adhesion to molten polymers, leading to an increase in the viscosity of the molten polymers and making it difficult to blend with the molten polymers.

Thus, there exists a need for approaches to improve the mechanical properties of conventional biodegradable polymers in terms of mechanical strength while maintaining the processing properties of the polymers as constant as possible.

### SUMMARY OF THE INVENTION

It is an object of the present invention to provide approaches to improve the mechanical properties of conventional biodegradable polymers in terms of mechanical strength while maintaining the processing properties of the polymers as constant as possible. It is a more specific object of the present invention to provide a biodegradable polymer whose physical properties are improved by the presence of a stereocomplex organic filler, and a method for producing the biodegradable polymer.

According to a representative aspect of the present invention, a biodegradable polymer is provided which includes (A) a stereocomplex organic filler and (B) a matrix polymer.

According to a further representative aspect of the present invention, a method for producing a biodegradable polymer is provided which includes (1) melting a matrix polymer or dissolving a matrix polymer in an organic solvent, (2) dispersing a stereocomplex organic filler in the melt or solution prepared in step (1), and (3) recovering the composite obtained in step (2).

According to another representative aspect of the present invention, a method for producing a stereocomplex organic filler is provided which includes (a) mixing poly-L-lactic acid and poly-D-lactic acid by a melting or supercritical fluid process to prepare a poly-L-lactic acid/poly-D-lactic acid composite, and (b) pulverizing the composite prepared in step (a).

According to exemplary embodiments of the present invention, the biodegradable polymer including a stereocomplex organic filler can find application in various fields, including biodegradable materials and medical materials, where conventional biodegradable polymers are difficult to use due to their inherent problems such as poor heat resistance and low strength.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a graph comparing the Young's moduli of biodegradable polymers including poly(lactic acid-co-glycolic acid) according to embodiments of the present invention as a function of the content of a stereocomplex organic filler;

FIG. 2 is a graph comparing the tensile strengths of biodegradable polymers including poly(lactic acid-co-glycolic acid) according to embodiments of the present invention as a function of the content of a stereocomplex organic filler;

FIG. 3 is a graph comparing the elongations at break of biodegradable polymers including poly(lactic acid-co-glycolic acid) according to embodiments of the present invention as a function of the content of a stereocomplex organic filler;

FIG. 4 is a graph comparing the Young's moduli of biodegradable polymers including poly(lactic acid) according to embodiments of the present invention with the Young's moduli of biodegradable polymers including commercial poly(lactic acid) (Lacty™) as a function of the content of a stereocomplex organic filler;

FIG. 5 is a graph comparing the tensile strengths of biodegradable polymers including poly(lactic acid) according to embodiments of the present invention with the tensile strengths of biodegradable polymers including commercial poly(lactic acid) (Lacty™) as a function of the content of a stereocomplex organic filler; and

FIG. 6 is a graph comparing the elongations at break of biodegradable polymers including poly(lactic acid) according to embodiments of the present invention with the elongations at break of biodegradable polymers including commercial poly(lactic acid) (Lacty™) as a function of the content of a stereocomplex organic filler.

### DETAILED DESCRIPTION OF THE INVENTION

Several aspects and embodiments of the present invention will now be described in more detail.

The term “biodegradable polymer” as used herein refers to a bioabsorbable polymeric material that is degraded in vivo.

According to one aspect of the present invention, a biodegradable polymer is disclosed which includes (A) a stereocomplex organic filler and (B) a matrix polymer.

The biodegradable polymer including a stereocomplex organic filler and a matrix polymer according to one embodiment of the present invention can find application in various fields, including biodegradable materials and medical materials, where conventional biodegradable polymers are difficult to use due to their inherent problems such as poor heat resistance and low strength.

In one embodiment of the present invention, the stereocomplex organic filler (A) is a poly-L-lactic acid/poly-D-lactic acid composite.

In a further embodiment of the present invention, the stereocomplex organic filler (A) is prepared by a melting process or using a supercritical fluid.

Preferably, the stereocomplex organic filler (A) is prepared using a supercritical fluid. Unlike the use of the stereocomplex organic filler (A) prepared using a supercritical fluid, the use of conventional stereocomplex organic fillers prepared using only an organic solvent such as chloroform or by a heating process cannot ensure advantageous effects in terms of elongation at break.

In another embodiment of the present invention, the ratio of the content of the poly-L-lactic acid to that of the poly-D-lactic acid in the stereocomplex organic filler (A) is from 2:8 to 8:2.

In a further embodiment of the present invention, the stereocomplex organic filler (A) is included in an amount of 0.1 to 5 parts by weight, based on 100 parts by weight of the matrix polymer (B).

According to one embodiment of the present invention, it is preferred to add the stereocomplex organic filler in a small amount of 0.1 to 5 parts by weight, based on 100parts by weight of the matrix polymer. If the amount of the stereocomplex organic filler added is less than the lower limit (i.e. 0.1 parts by weight), the problem of low mechanical strength may be caused. Meanwhile, if the amount of the stereocomplex organic filler added exceeds the upper limit (i.e. 5 parts by weight), the strength of the polymer after processing may be lowered.

In another embodiment of the present invention, the stereocomplex organic filler (A) has a size of 0.1 to 250 μm.

In one embodiment of the present invention, the size of the stereocomplex organic filler affects the mechanical properties of the biodegradable polymer. If the size of the stereocomplex organic filler is larger than 250 μm in size, the affinity of the stereocomplex organic filler for the matrix polymer may deteriorate.

In a further embodiment of the present invention, the stereocomplex organic filler (A) has a molecular weight of 3,000 to 500,000.

In another embodiment of the present invention, the stereocomplex organic filler (A) further includes, per 100 parts by weight thereof, 0.1 to 50 parts by weight of one or more homopolymers selected from polycaprolactone, polyglycolide, polycarbonate and polyethylene terephthalate, or a copolymer of the homopolymers.

In a further embodiment of the present invention, the matrix polymer (B) is selected from poly-L-lactic acid (PLLA), poly(lactic acid-co-glycolic acid) (PLGA), polyglycolic acid (PGA), polycaprolactone (PCL), and mixtures thereof.

The matrix polymer may be PLGA in which the ratio of LA to GA is 80:20. Biodegradable polymers using PLGA copolymers other than the PLGA copolymer whose LA:GA ratio is 80:20 may not exhibit the advantage of ease of molding processability.

In another embodiment of the present invention, the biodegradable polymer is used for a cardiovascular material selected from stents, surgical sutures, supports for tissue regeneration, bio-nanofibers, hydrogels and bio-sponges, a biomaterial for dentistry, neurosurgery, orthopedic surgery or plastic surgery selected from pins, screws and rods, or a material selected from drug and cell delivery vectors.

According to one embodiment of the present invention, due to the presence of the organic filler, the biodegradable polymer can provide a solution to the problems of conventional biodegradable polymers, such as problems in terms of processing, poor surface quality of molded parts and low mechanical strength, and is thus suitable for use as a medical material.

According to a further aspect of the present invention, a method for producing a biodegradable polymer is disclosed which includes (1) melting a matrix polymer or dissolving a matrix polymer in an organic solvent, (2) dispersing a stereocomplex organic filler in the melt or solution prepared in step (1), and (3) recovering the composite obtained in step (2).

According to one embodiment of the present invention, the dispersion of the stereocomplex organic filler in the matrix polymer can improve the overall physical properties of the matrix polymer.

Preferably, in step (2), ultrasonic waves are applied to increase the degree of dispersion of the stereocomplex organic filler. In step (3), the dispersion may be cast and heated to remove the organic solvent.

In another embodiment of the present invention, the matrix polymer is added in an amount of 10 to 20% by weight, based on the weight of the organic solvent.

In a further embodiment of the present invention, the organic solvent is selected from chloroform, toluene, xylene, dioxane, and tetrahydrofuran.

In another embodiment of the present invention, the stereocomplex organic filler includes poly-L-lactic acid and poly-D-lactic acid.

In a further embodiment of the present invention, the stereocomplex organic filler has a molecular weight of 3,000 to 500,000.

In another embodiment of the present invention, the stereocomplex organic filler is added in an amount of 0.1 to 5% by weight, based on the weight of the matrix polymer.

In a further embodiment of the present invention, the stereocomplex organic filler has a size of 0.1 to 250 μm.

According to another aspect of the present invention, a method for preparing the stereocomplex organic filler includes (a) mixing poly-L-lactic acid and poly-D-lactic acid by a melting or supercritical fluid process to prepare a poly-L-lactic acid/poly-D-lactic acid composite, and (b) pulverizing the composite prepared in step (a).

In one embodiment of the present invention, the supercritical fluid process may be carried out by mixing the poly-L-lactic acid and the poly-D-lactic acid in a supercritical fluid at a pressure of 100 to 500 bar and a temperature of 50 to 150° C.

In one embodiment of the present invention, the ratio of the content of the poly-L-lactic acid to that of the poly-D-lactic acid in the stereocomplex organic filler is from 2:8 to 8:2.

The present invention will be explained in more detail with reference to the following examples. However, these examples are not to be construed as limiting or restricting the scope and disclosure of the invention. It is to be understood that based on the teachings of the present invention including the following examples, those skilled in the art can readily practice other embodiments of the present invention whose experimental results are not explicitly presented.

### PREPARATIVE EXAMPLE 1

5 wt % of poly-L-lactic acid and 5 wt % of poly-D-lactic acid were mixed with 65 wt % of supercritical carbon dioxide and 25 wt % of methylene chloride, followed by stereocomplexation at a pressure of 350 bar and a temperature of 75° C. to prepare a stereocomplex poly-L-lactic acid/poly-D-lactic acid composite.

The stereocomplex composite was pulverized using a pulverizer and classified into different average particle diameters of 0.1-5 μm, 5-53 μm, 53-125 μm, and 125-212 μm before use.

### PREPARATIVE EXAMPLE 2

A stereocomplex composite was prepared in the same manner as in Preparative Example 1, except that chloroform was used instead of supercritical carbon dioxide.

### PREPARATIVE EXAMPLE 3

50 wt % of poly-L-lactic acid and 50 wt % of poly-D-lactic acid were dissolved by heating to a temperature of 180° C. The respective solutions were mixed, reacted with stirring, and cooled, affording a stereocomplex composite.

### PREPARATIVE EXAMPLE 4

100 wt % of L-lactide and 0.02 wt % of Sn(Oct)₂ were dissolved by heating to a temperature of 130° C., reacted with stirring for 24 h, and cooled, affording poly-L-lactic acid.

### EXAMPLE 1

Poly(lactic acid-co-glycolic acid) (PLGA) whose LA:GA ratio is 80:20 was dissolved in chloroform. The copolymer was used in an amount of 15 wt % with respect to the weight of the chloroform. The 0.1-5 μm sized stereocomplex composite prepared in Preparative Example 1 was dispersed in the chloroform solution by sonication. The stereocomplex composite was used in an amount of 0.5 wt % with respect to the weight of the PLGA. After completion of the reaction, the chloroform was volatilized to give a biodegradable polymer in the form of a film.

### EXAMPLE 2

A biodegradable polymer film was produced in the same manner as in Example 1, except that the stereocomplex composite was added in an amount of 1 wt %, based on the weight of the PLGA.

### EXAMPLE 3

A biodegradable polymer film was produced in the same manner as in Example 1, except that the stereocomplex composite was added in an amount of 2 wt %, based on the weight of the PLGA.

### EXAMPLE 4

A biodegradable polymer film was produced in the same manner as in Example 1, except that the 5-53 μm sized stereocomplex composite was used.

### EXAMPLE 5

A biodegradable polymer film was produced in the same manner as in Example 4, except that the stereocomplex composite was added in an amount of 1 wt %, based on the weight of the PLGA.

### EXAMPLE 6

A biodegradable polymer film was produced in the same manner as in Example 4, except that the stereocomplex composite was added in an amount of 2 wt %, based on the weight of the PLGA.

### EXAMPLE 7

A biodegradable polymer film was produced in the same manner as in Example 1, except that the 53-125 μm sized stereocomplex composite was used.

### EXAMPLE 8

A biodegradable polymer film was produced in the same manner as in Example 7, except that the stereocomplex composite was added in an amount of 1 wt %, based on the weight of the PLGA.

### EXAMPLE 9

A biodegradable polymer film was produced in the same manner as in Example 7, except that the stereocomplex composite was added in an amount of 2 wt %, based on the weight of the PLGA.

### EXAMPLE 10

A biodegradable polymer film was produced in the same manner as in Example 1, except that the 125-212 μm sized stereocomplex composite was used.

### EXAMPLE 11

A biodegradable polymer film was produced in the same manner as in Example 10, except that the stereocomplex composite was added in an amount of 1 wt %, based on the weight of the PLGA.

### EXAMPLE 12

A biodegradable polymer film was produced in the same manner as in Example 10, except that the stereocomplex composite was added in an amount of 2 wt %, based on the weight of the PLGA.

### EXAMPLE 13

A biodegradable polymer film was produced in the same manner as in Example 1, except that 15 wt % of poly-L-lactic acid (PLLA) was used instead of 15 wt % of poly(lactic acid-co-glycolic acid).

### COMPARATIVE EXAMPLE 1

A biodegradable polymer film was produced in the same manner as in Example 1, except that the stereocomplex composite was not added.

### COMPARATIVE EXAMPLE 2

A biodegradable polymer film was produced in the same manner as in Example 1, except that both the 53-125 μm sized stereocomplex composite and the 125-212 μm sized stereocomplex composite were used without separating them.

### COMPARATIVE EXAMPLE 3

A biodegradable polymer film was produced in the same manner as in Example 1, except that the stereocomplex composite was added in an amount of 3 wt %, based on the weight of the PLGA.

### COMPARATIVE EXAMPLE 4

A biodegradable polymer film was produced in the same manner as in Example 1, except that the stereocomplex composite was added in an amount of 5 wt %, based on the weight of the PLGA.

### COMPARATIVE EXAMPLE 5

A biodegradable polymer film was produced in the same manner as in Example 6, except that the stereocomplex composite was added in an amount of 3 wt %, based on the weight of the PLGA.

### COMPARATIVE EXAMPLE 6

The procedure of Example 6 was repeated except that the stereocomplex composite was added in an amount of 5 wt %, based on the weight of the PLGA.

### COMPARATIVE EXAMPLE 7

The procedure of Example 7 was repeated except that the stereocomplex composite was added in an amount of 3 wt %, based on the weight of the PLGA.

### COMPARATIVE EXAMPLE 8

The procedure of Example 7 was repeated except that the stereocomplex composite was added in an amount of 5 wt %, based on the weight of the PLGA.

### COMPARATIVE EXAMPLE 9

The procedure of Example 10 was repeated except that the stereocomplex composite was added in an amount of 3 wt %, based on the weight of the PLGA.

### COMPARATIVE EXAMPLE 10

The procedure of Example 10 was repeated except that the stereocomplex composite was added in an amount of 5 wt %, based on the weight of the PLGA.

### COMPARATIVE EXAMPLE 11

A biodegradable polymer film was produced in the same manner as in Example 13, except that the stereocomplex composite was not added.

### COMPARATIVE EXAMPLE 12

A biodegradable polymer film was produced in the same manner as in Example 16, except that the stereocomplex composite was not added.

### EXPERIMENTAL EXAMPLE 1

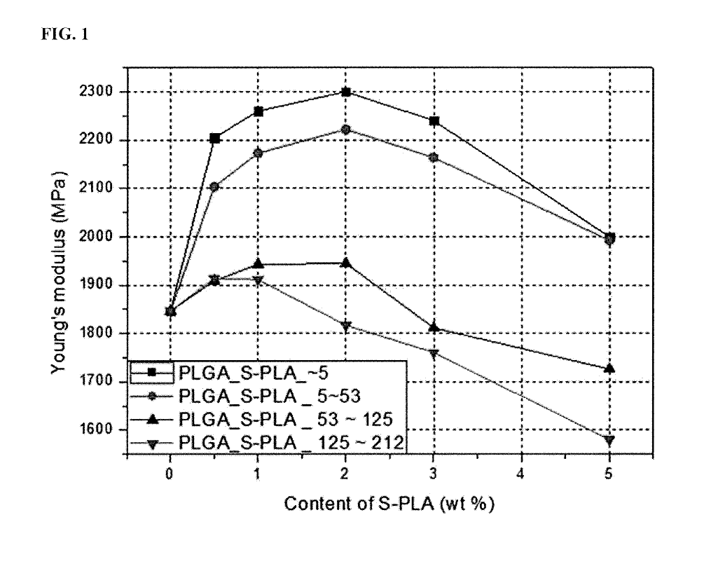

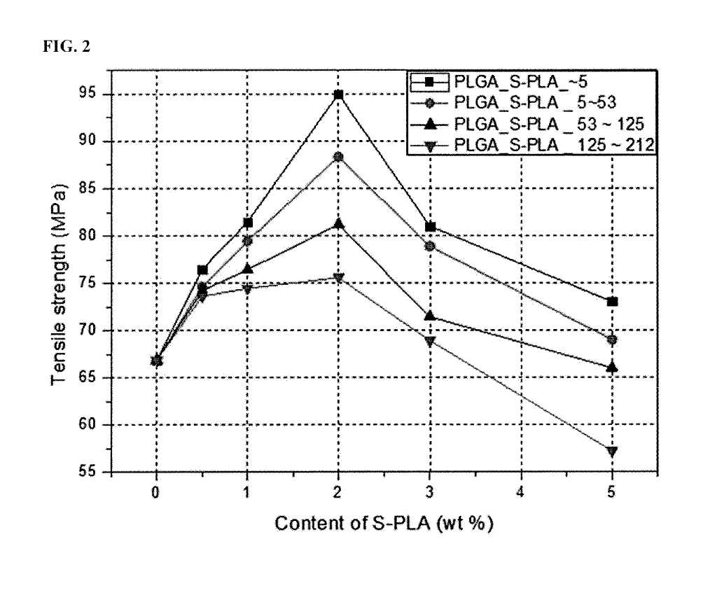

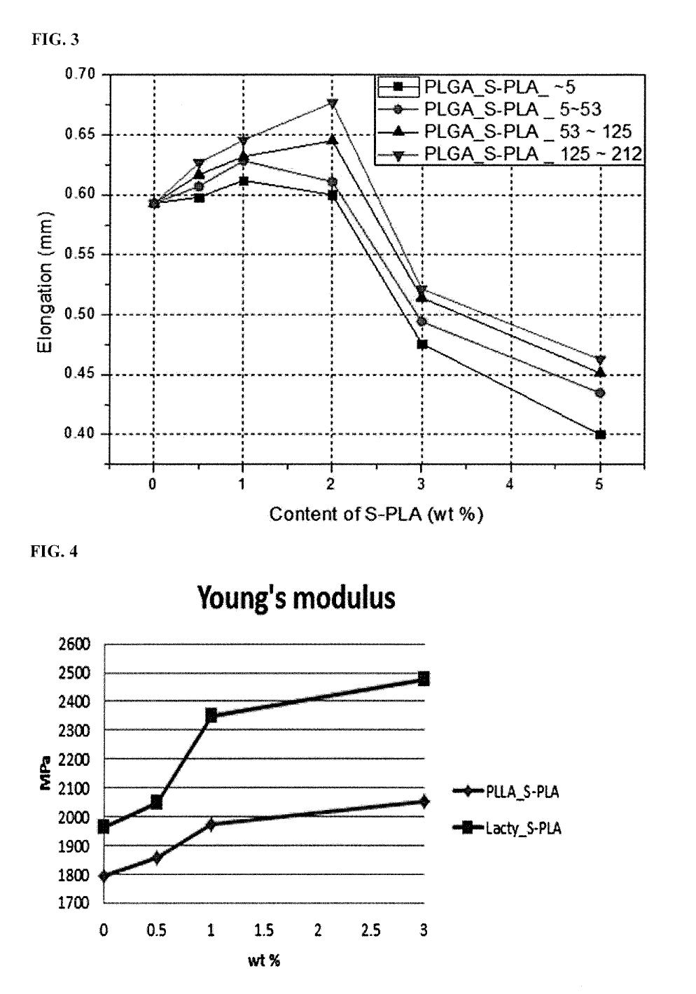

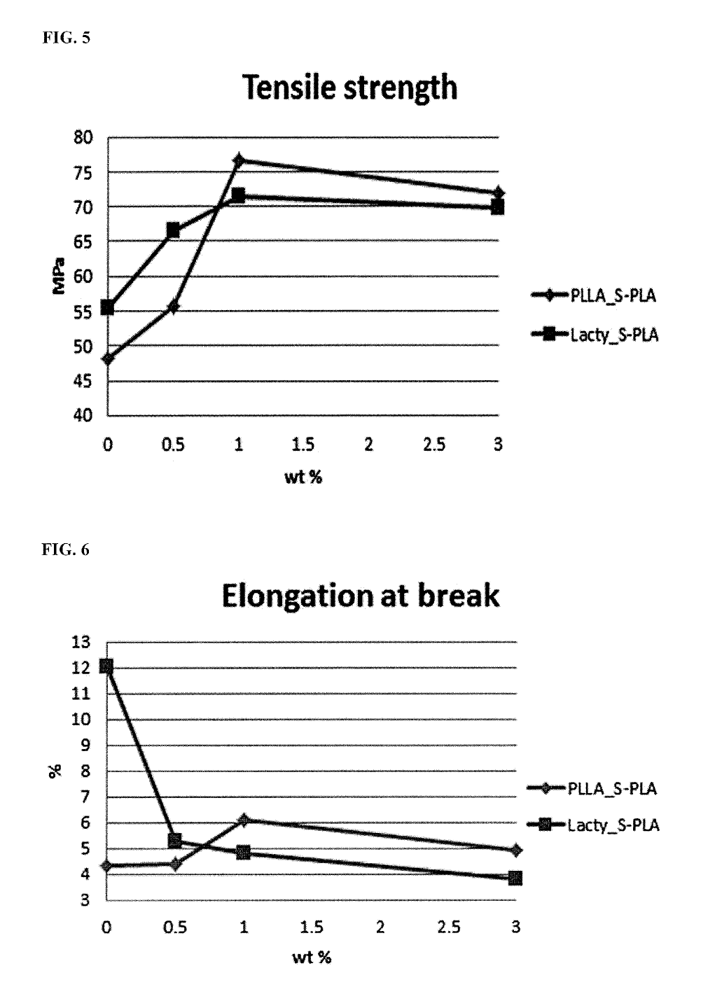

The Young's moduli, tensile strengths, and elongations at break of the biodegradable polymer films were measured using a universal testing machine (Instron model 4467, Canton, Mass., USA) to compare the mechanical strengths of the biodegradable polymers. The crosshead speed was maintained at 1 mm/min. The results are shown in Tables 1-6 and FIGS. 1-6.

TABLE 1
Comparison of Young's moduli of the biodegradable polymers
as a function of size and content of the stereocomplex composite

| | S-PLA organic filler | Young's modulus |
|---|---|---|
| Example No. | [Size (μm), content (wt %)] | (MPa) |
| Example 1 | 0.1-5, 0.5 | 2205 |
| Example 2 | 0.1-5, 1 | 2260 |
| Example 3 | 0.1-5, 2 | 2300 |
| Example 4 | 5-53, 0.5 | 2103 |
| Example 5 | 5-53, 1 | 2172 |
| Example 6 | 5-53, 2 | 2222 |
| Example 7 | 53-125, 0.5 | 1908 |
| Example 8 | 53-125, 1 | 1942 |
| Example 9 | 53-125, 2 | 1945 |
| Example 10 | 125-212, 0.5 | 1914 |
| Example 11 | 125-212, 1 | 1911 |
| Example 12 | 125-212, 2 | 1816 |
| Comparative Example 1 | — | 1846 |
| Comparative Example 2 | 53-125&125-212, 0.5 | 1720 |
| Comparative Example 3 | 0.1-5, 3 | 2240 |
| Comparative Example 4 | 0.1-5, 5 | 2000 |
| Comparative Example 5 | 5-53, 3 | 2163 |
| Comparative Example 6 | 5-53, 5 | 1992 |
| Comparative Example 7 | 53-125, 3 | 1811 |
| Comparative Example 8 | 53-125, 5 | 1726 |
| Comparative Example 9 | 125-212, 3 | 1760 |
| Comparative Example 10 | 125-212, 5 | 1579 |

TABLE 2
Comparison of tensile strengths of the biodegradable polymers
as a function of size and content of the stereocomplex composite

| | S-PLA organic filler | Tensile strength |
|---|---|---|
| Example No. | [Size (μm), content (wt %)] | (MPa) |
| Example 1 | 0.1-5, 0.5 | 76.4 |
| Example 2 | 0.1-5, 1 | 81.4 |
| Example 3 | 0.1-5, 2 | 95.2 |
| Example 4 | 5-53, 0.5 | 74.6 |
| Example 5 | 5-53, 1 | 79.4 |
| Example 6 | 5-53, 2 | 88.3 |
| Example 7 | 53-125, 0.5 | 74.1 |
| Example 8 | 53-125, 1 | 76.4 |
| Example 9 | 53-125, 2 | 81.1 |
| Example 10 | 125-212, 0.5 | 73.5 |
| Example 11 | 125-212, 1 | 74.4 |
| Example 12 | 125-212, 2 | 75.5 |
| Comparative Example 1 | — | 66 |
| Comparative Example 2 | 53-125&125-212, 0.5 | 65.5 |
| Comparative Example 3 | 0.1-5, 3 | 81.3 |
| Comparative Example 4 | 0.1-5, 5 | 73.5 |
| Comparative Example 5 | 5-53, 3 | 78.8 |
| Comparative Example 6 | 5-53, 5 | 68.9 |
| Comparative Example 7 | 53-125, 3 | 71.4 |
| Comparative Example 8 | 53-125, 5 | 65.9 |
| Comparative Example 9 | 125-212, 3 | 68.8 |
| Comparative Example 10 | 125-212, 5 | 57.2 |

TABLE 3
Comparison of elongations at break of the biodegradable polymers
as a function of size and content of the stereocomplex composite

| | S-PLA organic filler | Elongations at |
|---|---|---|
| Example No. | [Size (μm), content (wt %)] | break (%) |
| Example 1 | 0.1-5, 0.5 | 0.598 |
| Example 2 | 0.1-5, 1 | 0.612 |
| Example 3 | 0.1-5, 2 | 0.6 |
| Example 4 | 5-53, 0.5 | 0.607 |
| Example 5 | 5-53, 1 | 0.628 |
| Example 6 | 5-53, 2 | 0.610 |
| Example 7 | 53-125, 0.5 | 0.616 |
| Example 8 | 53-125, 1 | 0.631 |
| Example 9 | 53-125, 2 | 0.645 |
| Example 10 | 125-212, 0.5 | 0.626 |
| Example 11 | 125-212, 1 | 0.645 |
| Example 12 | 125-212, 2 | 0.676 |
| Comparative Example 1 | — | 0.592 |
| Comparative Example 2 | 53-125&125-212, 0.5 | 0.455 |
| Comparative Example 3 | 0.1-5, 3 | 0.476 |
| Comparative Example 4 | 0.1-5, 5 | 0.4 |
| Comparative Example 5 | 5-53, 3 | 0.494 |
| Comparative Example 6 | 5-53, 5 | 0.434 |
| Comparative Example 7 | 53-125, 3 | 0.514 |
| Comparative Example 8 | 53-125, 5 | 0.451 |
| Comparative Example 9 | 125-212, 3 | 0.521 |
| Comparative Example 10 | 125-212, 5 | 0.462 |

Tables 1-3 show the results of comparing the Young's moduli, tensile strengths, and elongations at break of the biodegradable polymer films as a function of the size of the organic filler. The strengths of the inventive biodegradable polymer films were at least about 1.4 times higher than the strength of the biodegradable polymer film including no organic filler. The strengths of the inventive biodegradable polymer films increased depending on the content of the organic filler. In addition, the biodegradable polymer films including the 125-212 μm sized organic filler showed higher mechanical strengths than the biodegradable polymer films including the 0.1-5 μm sized organic filler. Meanwhile, the biodegradable polymer film (Comparative Example 2) using both the 0.1-125 μm sized organic filler and the 125-212 μm sized organic filler was low in mechanical strength, particularly, elongation at break, indicating that the particle size of the organic filler has different influences on the mechanical strength of the film.

The mechanical properties shown in Tables 1-3 were collectively compared. As a result, the size of the organic filler is of importance in mechanical properties, and particularly, the films of Examples 3, 6, 9, and 12 including 1-2 wt % of the organic filler were confirmed to have the highest Young's moduli, tensile strengths, and elongations at break. When the organic filler content exceeded 3 wt %, the mechanical strengths of the films decreased considerably.

TABLE 4
Comparison of Young's moduli of the biodegradable polymers including
different kinds of matrix polymers

| | Kind of matrix polymer and | |
|---|---|---|
| | content (wt %) of S-PLA | Young's modulus |
| Example No. | organic filler (125-212 μm) | (MPa) |
| Example 7 | PLLA, 0.5 | 1865 |
| Example 8 | PLLA, 1 | 1990 |
| Example 9 | PLLA, 3 | 2050 |
| Example 10 | Lacty ™ (PLLA), 0.5 | 2050 |
| Example 11 | Lacty ™ (PLLA), 1 | 2345 |
| Example 12 | Lacty ™ (PLLA), 3 | 2480 |
| Comparative Example 7 | PLLA, 0 | 1800 |
| Comparative Example 8 | Lacty ™ (PLLA), 0 | 1980 |

TABLE 5
Comparison of tensile strengths of the biodegradable polymers including
different kinds of matrix polymers

| | Kind of matrix polymer and | |
|---|---|---|
| | content (wt %) of S-PLA | Tensile strength |
| Example No. | organic filler (125-212 μm) | (MPa) |
| Example 7 | PLLA, 0.5 | 56 |
| Example 8 | PLLA, 1 | 77 |
| Example 9 | PLLA, 3 | 74 |
| Example 10 | Lacty ™ (PLLA), 0.5 | 67 |
| Example 11 | Lacty ™ (PLLA), 1 | 72 |
| Example 12 | Lacty ™ (PLLA), 3 | 70 |
| Comparative Example 7 | PLLA, 0 | 47 |
| Comparative Example 8 | Lacty ™ (PLLA), 0 | 55 |

TABLE 6
Comparison of elongations at break of the biodegradable polymers
including different kinds of matrix polymers

| | Kind of matrix polymer and | |
|---|---|---|
| | S-PLA organic filler | Elongation at |
| Example No. | (125-212 μm) content (wt %) | break (%) |
| Example 7 | PLLA, 0.5 | 4 |
| Example 8 | PLLA, 1 | 6 |
| Example 9 | PLLA, 3 | 5 |
| Example 10 | Lacty ™ (PLLA), 0.5 | 5.2 |
| Example 11 | Lacty ™ (PLLA), 1 | 4.9 |
| Example 12 | Lacty ™ (PLLA), 3 | 3.8 |
| Comparative Example 7 | PLLA, 0 | 4 |
| Comparative Example 8 | Lacty ™ (PLLA), 0 | 12 |

As can be seen from the results in Tables 4-6, the addition of the organic filler increased the Young's modulus, tensile strength, and elongation at break of the biodegradable polymer films, demonstrating that the presence of the organic filler leads to an improvement in the strength of the biodegradable polymer films. The use of the conventional matrix polymer PLLA (Lacty™) showed a tendency to cause a decrease in elongation at break, which is believed to be because the conventional matrix polymer was partially crystallized.

### COMPARATIVE EXAMPLE 13

A biodegradable polymer film was produced in the same manner as in Example 13, except that the stereocomplex composite prepared in Preparative Example 2 was used.

### COMPARATIVE EXAMPLE 14

A biodegradable polymer film was produced in the same manner as in Example 13, except that the stereocomplex composite prepared in Preparative Example 3 was used.

The biodegradable polymer film of Example 1 was compared with the biodegradable polymer films of Comparative Examples 13 and 14. As a result, the use of the stereocomplex composite prepared in Preparative Example 1 was effective in achieving improved mechanical properties.

1-2. (canceled)

3. The biodegradable polymer according to claim 8, wherein the stereocomplex organic filler (A) is prepared by a melting or supercritical fluid process.

4. The biodegradable polymer according to claim 8, wherein the ratio of the content of the poly-L-lactic acid to that of the poly-D-lactic acid is from 2:8 to 8:2.

5. The biodegradable polymer according to claim 8, wherein the stereocomplex organic filler (A) is present in an amount of 0.1 to 5 parts by weight, based on 100 parts by weight of the matrix polymer (B).

6. The biodegradable polymer according to claim 8, wherein the stereocomplex organic filler (A) has a size of 0.1 to 250 μm.

7. The biodegradable polymer according to claim 8, wherein the stereocomplex organic filler (A) has a molecular weight of 3,000 to 500,000.

8. A biodegradable polymer comprising (A) a stereocomplex organic filler and (B) a matrix polymer, wherein the stereocomplex organic filter (A) is a poly-L-lactic acid/poly-D-lactic acid composite and further comprises, per 100 parts by weight thereof, 0.1 to 50 parts by weight of one or more homopolymers selected from the group consisting of polyglycolide, polycarbonate and polyethylene terephthalate, or a copolymer of the homopolymers; and wherein the matrix polymer (B) is selected from the group consisting of poly-L-lactic acid (PLLA), poly(lactic acid-co-glycolic acid)(PLGA), polyglycolic acid (PGA), polycaprolactone (PCL), and mixtures thereof.

9. The biodegradable polymer according to claim 8, wherein the matrix polymer (B) is poly-L-lactic acid (PLLA).

10. The biodegradable polymer according to claim 8, wherein the biodegradable polymer is used for a cardiovascular material selected from stents, surgical sutures, supports for tissue regeneration, bio-nanofibers, hydrogels and bio-sponges, a biomaterial for dentistry, neurosurgery, orthopedic surgery or plastic surgery selected from pins, screws and rods, or a material selected from drug and cell delivery vectors.

11. A method for producing a biodegradable polymer, comprising:
(1) melting a matrix polymer or dissolving a matrix polymer in an organic solvent;
(2) dispersing a stereocomplex organic filler in the melt or solution prepared in step (1); and
(3) recovering the composite obtained in step (2),
wherein the stereocomplex organic filler is a poly-L-lactic acid/poly-D-lactic acid composite and further comprises, per 100 parts by weight thereof, 0.1 to 50 parts by weight of one or more homopolymers selected from the group consisting of polyglycolide, polycarbonate and polyethylene terephthalate, or a copolymer of the homopolymers; and
wherein the matrix polymer is selected from the group consisting of poly-L-lactic acid (PLLA), poly(lactic acid-co-glycolic acid)(PLGA), polyglycolic acid (PGA), polycaprolactone (PCL), and mixtures thereof.

12. The method according to claim 11, wherein, in step (2), the stereocomplex organic filler is dispersed by the application of ultrasonic waves.

13. The method according to claim 11, wherein the stereocomplex organic filler comprises poly-L-lactic acid and poly-D-lactic acid.

14. The method according to claim 11, wherein the stereocomplex organic filler has a molecular weight of 3,000 to 500,000.

15. The method according to claim 11, wherein the stereocomplex organic filler is added in an amount of 0.1 to 5% by weight, based on the weight of the matrix polymer.

16. The method according to claim 11, wherein the stereocomplex organic filler has a size of 0.1 to 250 μm.

17. A biodegradable polymer comprising (A) a stereocomplex organic filler and (B) a matrix polymer, wherein the stereocomplex organic filler (A) is a poly-L-lactic acid/poly-D-lactic acid composite and further comprises, per 100 parts by weight thereof, 0.1 to 50 parts by weight of one or more homopolymers selected from the group consisting of polyglycolide, polycarbonate and polyethylene terephthalate, or a copolymer of the homopolymers; and wherein the matrix polymer (B) is poly-L-lactic acid (PLLA).

18. The biodegradable polymer according to claim 17, wherein the stereocomplex organic filler (A) is present in an amount of 0.1 to 5 parts by weight, based on 100 parts by weight of the matrix polymer (B).

19. The biodegradable polymer according to claim 17, wherein the stereocomplex organic filler (A) has a size of 0.1 to 250 μm.

20. The biodegradable polymer according to claim 17, wherein the stereocomplex organic filler (A) has a molecular weight of 3,000 to 500,000.
